(19) United States
(12) Patent Application Publication
(10) Pub. No.: US 2025/0346967 A1
(43) Pub. Date: Nov. 13, 2025

(54) PRIMER FOR AMPLIFYING MOLECULAR MARKER OF POWDERY MILDEW RESISTANCE GENE PMDR776 OF TRITICUM DICOCCON SCHRANK AND APPLICATION THEREOF

(71) Applicants: Crop Research Institute, Shandong Academy of Agricultural Sciences, Jinan (CN); Yantai University, Yantai (CN); National Center of Technology Innovation for Comprehensive Utilization of Saline-Alkali Land, Dongying (CN)

(72) Inventors: Cheng LIU, Jinan (CN); Pengtao MA, Yantai (CN); Yuli JIN, Yantai (CN); Ran HAN, Jinan (CN); Xiaolu WANG, Jinan (CN); Faji LI, Jinan (CN); Shengnan ZHAI, Jinan (CN); Haosheng LI, Jinan (CN); Jianjun LIU, Jinan (CN)

(21) Appl. No.: 19/017,426

(22) Filed: Jan. 11, 2025

(30) Foreign Application Priority Data
May 10, 2024 (CN) 2024105756714

(51) Int. Cl. C12Q 1/6895; A01H 1/04; C12Q 1/6806; C12Q 1/686; G01N 27/447

(52) U.S. Cl. CPC C12Q 1/6895; A01H 1/045; C12Q 1/6806; C12Q 1/686; G01N 27/44726; C12Q 2600/13

(57) ABSTRACT

The disclosure discloses a primer for amplifying a molecular marker of powdery mildew resistance gene PmDR776 of Triticum dicoccon Schrank, where the molecular marker primer includes a forward primer HENU654-F and a reverse primer HENU654-R; the nucleotide sequence of the forward primer HENU654-F is shown in SEQ ID NO:1, and the nucleotide sequence of the reverse primer HENU654-R is shown in SEQ ID NO:2. The molecular marker primer provided by the disclosure is applied to detection and identification of gene PmDR776 and auxiliary identification of a wheat powdery mildew resistance trait, and molecular marker-assisted selection breeding.

### TECHNICAL FIELD

The disclosure relates to the technical field of agricultural biological genes, and in particular to a primer for amplifying a molecular marker of powdery mildew resistance gene PmDR776 of Triticum dicoccon Schrank and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 25003TLAN-USP1-SL.xml. The XML file is 2,918 bytes; is created on Jan. 9, 2025; and is being submitted electronically via patent center.

### BACKGROUND

Wheat is the most widely cultivated crop in the world, with more than one-third of the global population relying on wheat products as their staple food (Appels et al. Shifting the limits in wheat research and breeding using a fully annotated reference genome. Science, 2018, 361: 661). Powdery mildew is one of the major diseases affecting wheat production. Measures to control powdery mildew in wheat mainly include chemical control and the breeding and promotion of resistant varieties. Compared with the traditional chemical control, the safe, economic and effective method for controlling wheat powdery mildew is to excavate the wheat variety with the disease resistance gene and breed the disease resistance variety by using the resistance source and the disease resistance gene.

In recent years, more than 100 wheat genes or their alleles conferring resistance to powdery mildew have been identified and molecularly mapped, including 64 officially named genes and dozens of provisionally named wheat powdery mildew resistance genes. In China's major wheat cultivars, the genetic diversity of genes conferring resistance to powdery mildew is relatively narrow, primarily consisting of a few resistance genes such as Pm2, Pm4b, Pm6, Pm21, and Pm52. Studies have shown that in various locations in Hebei Province and Gansu Province, powdery mildew strains toxic to Pm21 have emerged. The reliance on a single resistance gene indicates that its resistance capability is weakening. There are abundant excellent genes in wheat related species and genera, which hold significant value for the genetic improvement of wheat. These resources are not only utilizable for wheat breeding but also greatly expand the genetic base of wheat, providing strong support for enhancing its genetic diversity and disease resistance, among other aspects.

Triticum dicoccon Schrank originated from the northern part of fertile crescent soil, and has been widely spread in Europe, West Asia, Middle East, Northeast Africa and Southeast Asia for nearly 7,000 years (Maria et al. Triticum dicoccon Schrank (Triticum dicoccon Schrank), an old crop with promising future: a review. Genet Resour Crop Evol, 2010, 57: 937-962). Triticum dicoccon Schrank (Triticum dicoccon Schrank, 2n=28, AABB) belongs to the secondary gene pool of common wheat. Four wheat powdery mildew resistance genes (Pm4a, Pm5a, Pm49 and Pm50) were excavated from Triticum dicoccon Schrank. Moreover, Triticum dicoccon Schrank also shows excellent resistance against various diseases such as insect pests, stem rust and leaf rust, and also shows rich characteristics in the aspect of genetic diversity (Lhamo et al. Genome-wide association analyses of leaf rust resistance in Triticum dicoccon Schrank. Theoretical and Applied Genetics, 2023, 136:1). Therefore, it is necessary to find and locate new powdery mildew resistance genes in Triticum dicoccon Schrank of wheat related species genus and apply them to wheat powdery mildew resistance breeding so as to enrich wheat powdery mildew antigens.

### SUMMARY

The disclosure aims to provide a primer for amplifying a molecular marker of powdery mildew resistance gene PmDR776 of Triticum dicoccon Schrank and an application thereof, the primer amplification molecular marker is used for positioning and detecting the wheat powdery mildew resistance gene PmDR776, the parent of the wheat powdery mildew resistance gene PmDR776 is purposefully selected in wheat breeding, and a guidance basis is provided for breeding new wheat varieties resistant to powdery mildew.

The disclosure is realized by the following method: a primer for amplifying a molecular marker of powdery mildew resistance gene PmDR776 of Triticum dicoccon Schrank is provided, where the molecular marker primer includes a forward primer HENU654-F and a reverse primer HENU654-R; the nucleotide sequence of the forward primer HENU654-F is shown in SEQ ID NO:1, and the nucleotide sequence of the reverse primer HENU654-R is shown in SEQ ID NO:2.

The present disclosure provides an application of the primer for amplifying the molecular marker of powdery mildew resistance gene PmDR776 of Triticum dicoccon Schrank in detection and identification of gene PmDR776, auxiliary identification of a wheat powdery mildew resistance trait, and molecular marker-assisted selection breeding.

The disclosure further provides a method for detecting whether powdery mildew resistance gene PmDR776 of Triticum dicoccon Schrank is carried in a wheat sample, the method includes the following steps:
(1) extracting a genomic deoxyribonucleic acid (DNA) of a wheat sample to be detected;
(2) performing polymerase chain reaction (PCR) amplification on the genomic DNA of the wheat sample to be detected by utilizing a molecular marker primer to obtain an amplification product; and
(3) performing electrophoresis and detection on the amplification product, where if a 290 bp specific band is capable of being simultaneously amplified, the wheat sample to be detected is indicated to carry the powdery mildew resistance gene PmDR776 of Triticum dicoccon Schrank, otherwise, the wheat sample to be detected is indicated not to carry the powdery mildew resistance gene PmDR776 of Triticum dicoccon Schrank.

In the method, the molecular marker primer includes a forward primer HENU654-F and a reverse primer HENU654-R;
the nucleotide sequence of the forward primer HENU654-F is 5′-GTGGCAGTGCTTGGAAGTGAT-3′, as shown in SEQ ID NO:1; and
the nucleotide sequence of the reverse primer HENU654-R is 5′-ACGTCGATTTGGGAAGTTTAG-3′, as shown in SEQ ID NO:2.

A PCR amplification system in step (2) is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification in step (2) is: performing predenaturation for 3 minutes at 94° C., performing denaturation for 15 seconds at 94° C., performing annealing for 20 seconds at 55° C., performing extension for 40 seconds, and performing 30 cycles; performing extension for 10 minutes at 72° C.; and performing preservation at 4° C.

A procedure of the electrophoresis of the amplification product in step (3) is: performing electrophoresis on a non-denaturing polyacrylamide gel with the mass volume percentage concentration of 8%, after the amplification product is mixed with 2 μL of 6× sample loading buffer, taking 2 μL of the mixture for spot sampling, performing electrophoresis under constant pressure of 180 V for 2.5-3 hours, and performing photographing after silver nitrate staining.

Triticum dicoccon Schrank DR776 used in the research of the present disclosure has high powdery mildew resistance in the seedling stage and the adult plant stage, and genetic analysis and molecular marker detection of powdery mildew resistance in the seedling stage show that the resistance of Triticum dicoccon Schrank DR776 to wheat powdery mildew with different toxicity in the seedling stage is controlled by a pair of dominant genes PmDR776, which are mapped on a wheat chromosome 2AL, and are a novel powdery mildew resistance gene/allele of wheat. Through genetic segregation population detection, the molecular marker HENU654 of the powdery mildew resistance gene PmDR776 of wheat provided by the present disclosure is found to have a genetic distance of 1.21 cM from the gene PmDR776, and be closely linked to the gene PmDR776, which can be used for accurately detecting a large genetic mapping population of the gene PmDR776, and the gene PmDR776 could be located, detected, identified and assisted in the identification of wheat powdery mildew resistance traits, fine positioning, map cloning and molecular marker-assisted breeding. The disclosure provides a molecular marker primer closely linked with a powdery mildew resistance gene PmDR776 of wheat, which is applied to powdery mildew resistance wheat breeding, such that target varieties are quickly and accurately screened, which is not affected by an environment, the selection target is clear, the production cost is saved, and the selection efficiency and quality of high-quality powdery mildew-resistant wheat varieties or strains are greatly improved, and the molecular marker primer is of great significance for realizing the breeding of powdery mildew resistance wheat varieties and effectively preventing and controlling wheat powdery mildew.

### BRIEF DESCRIPTION OF THE DRAWING

FIG. 1 shows some results of detection of Triticum dicoccon Schrank DR776 and Triticum turgidum Langdon as well as segregation populations of hybrids thereof through a primer of a molecular marker HENU654.

In the figure, M: DL2000; 1: Triticum dicoccon Schrank DR776 (powdery mildew resistance variety carrying the gene PmDR776); 2: Triticum turgidum Langdon (powdery mildew susceptible variety); 3-22: F_{2:3} families formed by crossing the Triticum dicoccon Schrank DR776 with the Triticum turgidum Langdon, where 3-7: F_{2:3} family homozygous for resistance, 8-17: F₂:3 family for resistance and susceptibility segregation, and 18-22: F_{2:3} family homozygous for susceptibility; and band indicated by a white arrow is specific band tracing the gene PmDR776.

### DETAILED DESCRIPTION OF THE EMBODIMENTS

The following embodiments are used for understanding the present disclosure better, but not intended to limit the present disclosure. Experimental methods in the following embodiments are conventional methods unless otherwise specified. All experimental materials, reagents, etc. used in the embodiments may be obtained from a commercial approach unless otherwise specified.

Embodiment 1 Development of molecular marker HENU654 primer of powdery mildew resistance gene PmDR776 of wheat

### Materials

Triticum dicoccon Schrank DR776 and Triticum turgidum Langdon are resistant and susceptible to wheat powdery mildew respectively. F₁ hybrids of the Triticum dicoccon Schrank DR776 and the Triticum turgidum Langdon are selfed to obtain a F₂ population and corresponding F_{2:3} families.

### Genomic DNA Extraction of Wheat

A cetyltrimethylammonium bromide (CTAB) method for genomic DNA extraction of wheat includes the process as follows:

(1) Fresh wheat leaves are cut and ground with liquid nitrogen about 0.5 g of ground wheat leaves is put into a 2.0 mL centrifuge tube.

(2) 600 μL of CTAB extract solution is added and placed in a water bath at 65° C. for 40 minutes, where mixing up and down is performed once every 10 minutes.

(3) 600 μL of chloroform-isoamyl alcohol (24:1, v/v) is added and shaken gently on a shaking table for 15 minutes.

(4) Centrifugation is performed at 12000 rpm for 15 minutes, supernatant is taken to another 2 mL centrifuge tube, 3 times volume of precooled absolute ethanol is added, and precipitation is performed at a refrigerator at −20° C. for 60 minutes.

(5) Flocculent DNA precipitate is picked out and washed twice with 75% precooled ethanol.

(6) The DNA precipitate is picked out, placed in a 1.5 mL centrifuge tube, and air-dried indoors.

(7) 60 μL of TE buffer (100 mM Tris-Hcl, 10 mM EDTA, pH=8.0) is added to dissolve the DNA precipitate to prepare a DNA storage solution.

(8) The DNA storage solution is diluted to 20-30 ng/μL with ultrapure water as a working solution for later use.

### 3. Identification and Genetic Analysis of Powdery Mildew Resistance in Wheat Seedling Stage

The resistance parent Triticum dicoccon Schrank DR776, the susceptible parent Triticum turgidum Langdon, the hybrid F₁, the F₂ population and the F_{2:3} families are planted in a 128-hole tray (2×2 cm), 5 seeds are sown in each hole, 20 seeds of each of the patents and F₁ are identified, 20 seeds of each F_{2:3} family are sown, and a susceptible control Mingxian 169 is sown randomly and labeled. After sowing, growth conditions are controlled as a 14 hours light/10 hours darkness cycle, a temperature of 20° C., and relative humidity of 30-40%. When the seedlings grow to a one leaf stage, and are inoculated with a wheat powdery mildew strain E09. The conditions for the first 24 hours after inoculation are controlled as follows: darkness, a temperature of 20° C., and relative humidity of 100%. Then, the conditions are controlled as follows: a 14 hours light/10 hours darkness cycle, a temperature of 18-22° C., and relative humidity of 100%. When the first leaf of the susceptible control Mingxian 169 is fully infected, phenotypes are recorded according to the standards of grades 0-4, where grades 0-2 are regarded as resistance grades, and grades 3 and 4 are regarded as susceptible grades.

Investigation results show that the Triticum dicoccon Schrank DR776 is immune to the strain E09, and the Triticum turgidum Langdon (LDN) is highly susceptible to the strain E09. Hybrid F₁ between the Triticum dicoccon Schrank DR776 and the Triticum turgidum Langdon shows resistance. 112 of 151 F₂ plants show resistance, and 32 show susceptibility, which accords with a segregation ratio of resistance to susceptibility of 3:1. Among 151 F_{2:3} families, 37 are homozygous for resistance, 75 are heterozygous, 39 are homozygous for susceptibility, and the segregation ratio of homozygous resistance: heterozygosis: homozygous susceptibility is 1:2:1. Therefore, the resistance of the Triticum dicoccon Schrank DR776 to the strain E09 is controlled by a single dominant gene, named gene PmDR776.

### 4. Initial Mapping of Molecular Marker of Wheat Powdery Mildew Resistance Gene PmDR776

According to phenotypic identification results, 20 homozygous resistance families and 20 homozygous susceptible families are selected to construct a resistance pool and a susceptible pool respectively. Polymorphism detection is performed on the Triticum dicoccon Schrank DR776, the Triticum turgidum Langdon, the resistance pool and the susceptible pool by using genome-wide evenly distributed molecular markers. 10 pairs of markers show consistent polymorphism in the resistance and susceptible parents and the resistance and susceptible pools. Then, 151 F_{2:3} families of Triticum dicoccon Schrank DR776×Triticum turgidum Langdon are genotyped by using these markers, and the gene PmDR776 is initially mapped at a terminal of the wheat chromosome 2AL.

### 5. Development of Markers Closely Linked to Gene PmDR776

According to sequence information of the reference genome cv. Svevo of the Triticum dicoccum, simple sequence repeat (SSR) markers are designed through software primer5.0. The F_{2:3} families of Triticum dicoccon Schrank DR776×Triticum turgidum Langdon are genotyped, the SSR marker HENU654 closely linked to the gene PmDR776 is obtained, and a genetic distance is only 1.21 cM.

A primer of the molecular marker HENU654 includes a forward primer and a reverse primer:
a nucleotide sequence of the forward primer HENU654-F: 5′-GTGGCAGTGCTTGGAAGTGAT-3′(as shown in SEQ ID NO:1);

a nucleotide sequence of the reverse primer HENU654-R: 5′-ACGTCGATTTGGGAAGTTTAG-3′ is shown in SEQ ID NO:2.

A PCR amplification system is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification is: predenaturation for 3 minutes at 94° C., denaturation for 15 seconds at 94° C., annealing for 20 seconds at 55° C., extension for 40 seconds, and 30 cycles; extension for 10 minutes at 72° C.; and preservation at 4° C.

A procedure of electrophoresis of an amplification product is: electrophoresis is performed on a non-denaturing polyacrylamide gel with the mass volume percentage concentration of 8%, after the amplification product is mixed with 2 μL of 6× sample loading buffer, 2 μL of the mixture is taken for spot sampling, electrophoresis is performed under constant pressure of 180 V for 2.5-3 hours, and photographing is performed after silver nitrate staining.

If a specific band of 290 bp is simultaneously amplified, and wheat to be detected is indicated to carry the wheat powdery mildew resistance gene PmDR776; and if a specific band of 290 bp fails to be simultaneously amplified, and the wheat to be detected is indicated not to carry the wheat powdery mildew resistance gene PmDR776.

### Embodiment 2 Application of Molecular Marker HENU654 Primer of Wheat Powdery Mildew Resistance Gene PmDR776

Triticum dicoccon Schrank DR776, Triticum turgidum Langdon and materials in F₂:3 families formed by crossing the Triticum dicoccon Schrank DR776 with the Triticum turgidum Langdon are detected by the molecular marker HENU654 of the wheat powdery mildew resistance gene PmDR776, and a sample DNA extraction method is the same as that in Embodiment 1.

Samples to be detected: Triticum dicoccon Schrank DR776, Triticum turgidum Langdon and F_{2:3} families formed by crossing the Triticum dicoccon Schrank DR776 with the Triticum turgidum Langdon.

Genomic DNAs of the above materials are extracted as a template of PCR amplification, and the amplification is performed through the primer of the molecular marker HENU654 developed by the present disclosure:

nucleotide sequence of forward primer HENU654-F:
5′- GTGGCAGTGCTTGGAAGTGAT-3′;
(as shown in SEQ ID NO: 1)
nucleotide sequence of reverse primer HENU654-R:
5′-ACGTCGATTTGGGAAGTTTAG-3′.
(as shown in SEQ ID NO: 2)

A PCR amplification system is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

A procedure of the PCR amplification is: predenaturation for 3 minutes at 94° C., denaturation for 15 seconds at 94° C., annealing for 20 seconds at 55° C., extension for 40 seconds, and 30 cycles; extension for 10 minutes at 72° C.; and preservation at 4° C.

A procedure of electrophoretic segregation of an amplification product is: electrophoresis is performed on a non-denaturing polyacrylamide gel with the mass volume percentage concentration of 8%, after the amplification product is mixed with 2 μL of 6× sample loading buffer, 2 μL of the mixture is taken for spot sampling, electrophoresis is performed under constant pressure of 180 V for 2.5-3 hours, and photographing is performed after silver nitrate staining.

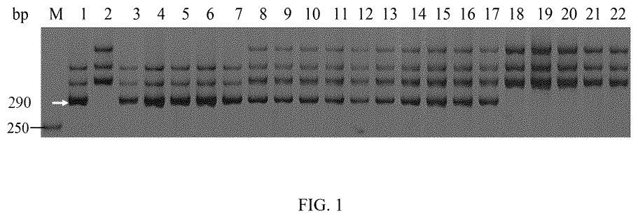

The results of molecular marker detection are shown in FIG. 1. FIG. 1 shows some amplification results of the marker HENU654 in F_{2:3} family derived from Triticum dicoccon Schrank DR776×Triticum turgidum Langdon. In the FIG. 1, M: DL2000; 1: Triticum dicoccon Schrank DR776 (powdery mildew resistance variety carrying the gene PmDR776); 2: Triticum turgidum Langdon (powdery mildew susceptible variety); 3-22: F_{2:3} families formed by crossing the Triticum dicoccon Schrank DR776 with the Triticum turgidum Langdon, where 3-7: F₂:3 family homozygous for resistance, 8-17: F_{2:3} family for resistance and susceptibility segregation, and 18-22: F_{2:3} family homozygous for susceptibility; and band indicated by a white arrow is specific band tracing the gene PmDR776. The amplification results show that the specific band of 290 bp of the marker HENU654 is amplified in the disease-resistant parent Triticum dicoccon Schrank DR776 and the disease-resistant families, but not in the susceptible parent Triticum turgidum Langdon and the susceptible families.

The gene PmDR776 originated from the Triticum dicoccon Schrank DR776, and mapping and map-based cloning of the gene has not been reported yet. The molecular marker HENU654 and the primer provided by the present disclosure are used for detecting a large genetic mapping population, which contributes to fine mapping and map-based cloning of the gene PmDR776, and has very important significance for efficient transformation of the gene PmDR776 and in-depth analysis of a disease resistance mechanism.

The above embodiments are optimized implementation plans of the present disclosure, and are only used for illustrating the present disclosure, not limiting the present disclosure. All modifications or equivalent substitutions made by those skilled in the art without departing from the purpose and principle of the implementation plans of the present disclosure fall within the scope claimed to be protected by the present disclosure.

What is claimed is:

1. A primer for amplifying a molecular marker of powdery mildew resistance gene PmDR776 of Triticum dicoccon Schrank, wherein the molecular marker primer comprises a forward primer HENU654-F and a reverse primer HENU654-R, wherein the nucleotide sequence of the forward primer HENU654-F is shown as SEQ ID NO:1, and the nucleotide sequence of the reverse primer HENU654-R is shown as SEQ ID NO:2.

2. The application of the primer for amplifying the molecular marker of powdery mildew resistance gene PmDR776 of Triticum dicoccon Schrank according to claim 1 in detection and identification of gene PmDR776, auxiliary identification of a wheat powdery mildew resistance trait, and molecular marker-assisted selection breeding.

3. A method for detecting whether powdery mildew resistance gene PmDR776 of Triticum dicoccon Schrank is carried in a wheat sample, comprising the following steps:
(1) extracting a genomic deoxyribonucleic acid (DNA) of a wheat sample to be detected;
(2) performing polymerase chain reaction (PCR) amplification on the genomic DNA of the wheat sample to be detected by utilizing a molecular marker primer to obtain an amplification product; and
(3) performing electrophoresis and detection on the amplification product, where if a 290 bp specific band is capable of being simultaneously amplified, the wheat sample to be detected is indicated to carry the powdery mildew resistance gene PmDR776 of Triticum dicoccon Schrank, otherwise, the wheat sample to be detected is indicated not to carry the powdery mildew resistance gene PmDR776 of Triticum dicoccon Schrank.

4. The method according to claim 3, wherein a PCR amplification system in step (2) is 10 μL, including: 1.0 μL of wheat genomic DNAs of 50 ng/μL, 5 μL of a PCR Master Mix, 0.4 μL of a forward primer of 5 μM, 0.4 μL of a reverse primer of 5 μM, and 3.2 μL of sterile deionized water.

5. The method according to claim 3, wherein the procedure of the PCR amplification in step (2) is: performing predenaturation for 3 minutes at 94° C., performing denaturation for 15 seconds at 94° C., performing annealing for 20 seconds at 55° C., performing extension for 40 seconds, and performing 30 cycles; performing extension for 10 minutes at 72° C.; and performing preservation at 4° C.


SEQUENCE LISTING (2 sequences)

SEQ ID NO: 1 - DNA, 21 bp, synthetic construct
  source 1..21: mol_type = other DNA
gtggcagtgcttggaagtgat

SEQ ID NO: 2 - DNA, 21 bp, synthetic construct
  source 1..21: mol_type = other DNA
acgtcgatttgggaagtttag